(19) United States
(12) Patent Application Publication
(10) Pub. No.: US 2023/0251200 A1
(43) Pub. Date: Aug. 10, 2023

(54) DETACHABLE MIXING DEVICE FOR REPRODUCIBLE ATP REACTION OF COMMERCIAL ATP OPTICAL MEASURING INSTRUMENT

(71) Applicant: UIF (University Industry Foundation), Yonsei University, Seoul (KR)

(72) Inventors: Jung Ho HWANG, Seoul (KR); Do Heon KIM, Seoul (KR); Sang Mo KANG, Gyeonggi-do (KR)

(73) Assignee: UIF (University Industry Foundation), Yonsei University, Seoul (KR)

(21) Appl. No.: 18/106,521

(22) Filed: Feb. 7, 2023

(30) Foreign Application Priority Data
Feb. 7, 2022 (KR) 10-2022-0015284

(51) Int. Cl. G01N 21/64; G01N 1/38

(52) U.S. Cl. CPC G01N 21/6486; G01N 1/38

(57) ABSTRACT

The present invention relates to a detachable mixing device for a reproducible adenosine triphosphate (ATP) reaction of a commercial ATP optical measuring instrument and provides a mixing device for an ATP optical measuring instrument, the mixing device including a main body, a swab kit insertion part which is formed in the main body and provides an insertion space for an ATP reaction swab kit, a mixing module which is disposed around a lower perimeter of the swab kit insertion part and mixes a reactant in the inserted swab kit, and a coupling part disposed on one side surface of the main body and detachably attached to the ATP optical measuring instrument.

### RELATED APPLICATION

This application claims the benefit of priority of Korean Patent Application No. 10-2022-0015284 filed on Feb. 7, 2022, the contents of which are incorporated by reference as if fully set forth herein in their entirety.

### FIELD AND BACKGROUND OF THE INVENTION

The present invention relates to a detachable mixing device for a reproducible adenosine triphosphate (ATP) reaction of a commercial ATP optical measuring instrument.

In conventional technology for enhancing a reactivity of bacteria detection based on an adenosine triphosphate (ATP)—luciferin reaction, there is a technology for reacting internal ATP with luciferin, wherein the internal ATP is extracted by breaking cell walls of bacteria with bullet particles generated by spark discharge of an aero-sniping-based rapid pathogen aerosol detection sensor. The above technology uses a method in which a high voltage is applied between two metal electrodes to form a conductive line between the two electrodes so as to generate the spark discharge, and surfaces of the electrodes are locally heated at high temperatures by sparks to vaporize an electrode material to generate nanoparticles used as the bullet particles. Due to continuous consumption of tellurium (Te) and silver (Ag) which are used as nanoparticles and a high voltage use condition therefor, there are disadvantages in terms of portability, safety, and economy.

### SUMMARY OF THE INVENTION

The present invention is directed to providing a detachable mixing device for a reproducible adenosine triphosphate (ATP) reaction of a commercial ATP optical measuring instrument.

According to an aspect of the present invention, there is provided a mixing device for an ATP optical measuring instrument, the mixing device comprising a main body; a swab kit insertion part which is formed in the main body and provides an insertion space for an ATP reaction swab kit; a mixing module which is disposed around a lower perimeter of the swab kit insertion part and mixes a reactant in the inserted swab kit; and a coupling part disposed on one side surface of the main body and detachably attached to the ATP optical measuring instrument.

In the present invention, the mixing module may be a vibration module.

In the present invention, the mixing module may be a sonicator.

In the present invention, the mixing module may be a vortexing module.

In the present invention, a speed of the mixing module may be in the range of 3,000 to 5,000 rpm.

In the present invention, the coupling part may include a coupling groove to be fitted onto the ATP optical measuring instrument.

In the present invention, the mixing device for the ATP optical measuring instrument may further include a battery mounted on the main body.

According to another aspect of the present invention, there is provided an ATP optical measuring system comprising an ATP optical measuring instrument; a swab; an ATP reaction swab kit; and the above-mentioned mixing device.

According to still another aspect of the present invention, there is provided an ATP optical measuring method comprising the steps of: attaching the mixing device to the ATP optical measuring instrument of the above-mentioned ATP optical measuring system; sampling a sample using a swab from a place where the presence or absence of bacteria is to be diagnosed; putting the swab with the sample thereon into the ATP reaction swab kit; mixing after inserting the swab kit, into which the swab is put, into the mixing device; fitting the mixed swab kit onto the ATP optical measuring instrument; measuring an intensity of light generated by an ATP-luciferin reaction; and checking a relative light unit (RLU) value which is a unit that represents an extent of the reaction.

The present invention provides a detachable vibration/sonicator/vortexing device for helping an ATP-luciferin reaction when a commercial ATP optical measuring instrument is used, thereby having a higher performance than the above-mentioned conventional technology in terms of portability, safety, and economy.

Since the conventional method of helping a smooth ATP-luciferin reaction is shaking manually, an RLU-luciferin reaction for the same sample cannot be constant, and thus quantitative comparison is difficult.

However, when the vibration/sonicator/vortexing device dedicated for a swab kit detachable to the commercial ATP optical measuring instrument according to the present invention is used, a constant rpm leads to the more constant ATP-luciferin reaction, thus quantitative comparison is possible.

In addition, a mixing device according to the present invention has a structure which is customized to be detachably attached to the commercial ATP optical measuring instrument, thereby having the convenient structure to be used together with the commercial ATP optical measuring instrument.

### BRIEF DESCRIPTION OF THE SEVERAL VIEWS OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a view illustrating an adenosine triphosphate (ATP) measuring system for bacteria;

FIG. 2 is a view illustrating a method of using an existing commercial ATP optical measuring instrument;

FIG. 3 is a set of views illustrating a mixing device for an ATP optical measuring instrument according to the present invention, wherein FIG. 3A is a plan view of the mixing device, and FIG. 3B is a front view of the mixing device;

FIGS. 4A-B are a set of graphs showing a reaction time versus a relative light unit (RLU) according to the presence or absence of mixing; and

FIG. 5 is a graph showing the highest RLU according to the presence or absence of mixing of samples having different concentrations.

### DESCRIPTION OF SPECIFIC EMBODIMENTS OF THE INVENTION

Hereinafter, the present invention will be described in detail with reference to the accompanying drawings.

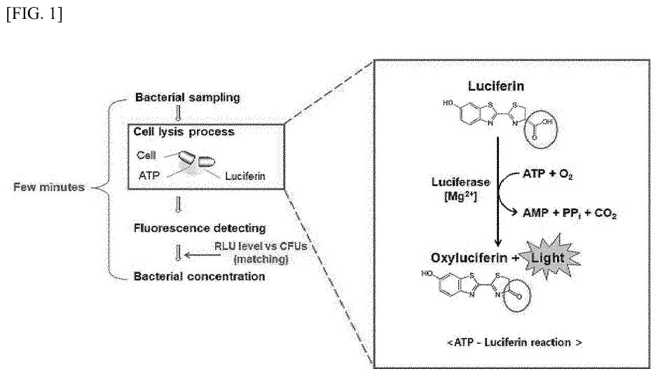

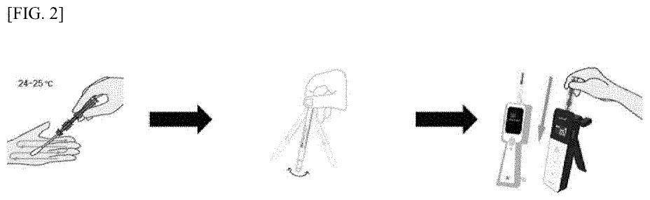

Referring to FIG. 1, as one of methods of detecting bacteria, there is a method of measuring an intensity of light generated by an adenosine triphosphate (ATP)—luciferin reaction based on an ATP bioluminescence reaction. When compared to other methods of detecting bacteria, this method can more easily and rapidly check the presence or absence of bacteria and an extent of the bacteria. In addition, since there are commercial ATP optical measuring instruments (made by, for example, 3M Co., Kikkoman Corp., and Charm Co.) manufactured by applying this method of detecting bacteria, anyone who has the corresponding instrument can easily sample and detect bacteria.

Referring to FIG. 2, when the corresponding commercial products are used, a procedure of three steps are mainly performed, wherein the three steps include a first step of sampling a sample using a commercial swab from a place where the presence or absence of bacteria is to be diagnosed, a second step of putting the swab with the sample thereon into an ATP reaction swab kit and manually shaking the ATP reaction swab kit to help an ATP reaction, and a final third step of fitting the reacted swab kit into an ATP optical measuring instrument and measuring an intensity of light generated by an ATP-luciferin reaction to check a relative light unit (RLU) value which is a unit that represents an extent of the reaction.

Among the above steps, since the “shaking” corresponding to the second step is a work performed by hands of human, a mixing extent cannot be constant, an extent or a speed of the ATP-luciferin reaction varies, and thus reproducibility is decreased even if the same RLU value is measured for the samples.

In the present invention, in order to solve this problem, by using a device for mixing through vibrating, sonicating, or vortexing at the same rpm (3,000 to 5,000 rpm), more reproducible detection is possible.

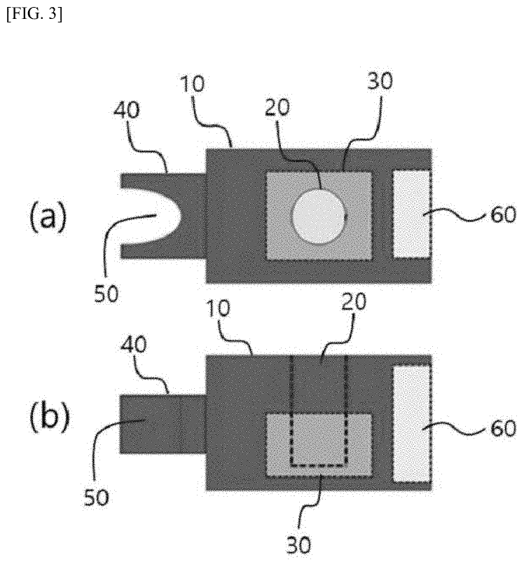

Referring to FIG. 3, a mixing device for an ATP optical measuring instrument according to the present invention may include a main body 10, a swab kit insertion part 20, a mixing module 30, a coupling part 40, a coupling groove 50, a battery 60, and the like.

The main body 10 forming a base frame of the mixing device may be formed of plastic or the like. For example, the main body 10 may be formed as a hexahedron having a length of 4 to 6 cm, a width of 3 to 5 cm, and a height of 5 to 6 cm.

The swab kit insertion part 20 serves to provide an insertion space of an ATP reaction swab kit. The swab kit insertion part 20 may include an open entrance formed in a central portion of an upper surface of the main body 10 and a spatial part extending downward from the entrance. The swab kit insertion part 20 may have a shape and a size corresponding to the swab kit and may be formed in a long cylindrical shape having, for example, a diameter of 0.8 to 1 cm and a height of about 4 cm.

The mixing module 30 serves to mix a reactant in the swab kit inserted into the swab kit insertion part 20. The mixing module 30 may be embedded in the main body 10 and disposed around a lower perimeter of the swab kit insertion part 20 to surround the swab kit insertion part 20.

The mixing module 30 may be a vibration module, a sonicator, or a vortexing module. The vibration module may be a vibrator or a vibration motor. The sonicator is a mixing module using an ultrasonic wave. The vortexing module is a shaking and mixing module through strong vibration. A speed of the mixing module may be, for example, in the range of 3,000 to 5,000 rpm. The mixing module 30 may be automatically operated when the swab kit is pushed from above (touch mode), and alternatively, a separate switch may be disposed on the main body 10, and the mixing module 30 may be operated by turning the switch on/off.

The coupling part 40 serves to be detachably attached to the ATP optical measuring instrument. The coupling part 40 may be disposed on one side surface of the main body 10 and have a smaller size than the main body 10 and integrally manufactured with the main body 10 or separately manufactured and fixed to the main body 10 using a screw or the like. The coupling part 40 may be formed as a hexahedron having a height of, for example, 2 to 3 cm.

The coupling groove 50 is a groove formed in the coupling part 40 and serves to be fitted onto the ATP optical measuring instrument. The coupling groove 50 may have a shape and a size corresponding to a part (case or the like) of the ATP optical measuring instrument so as to be exactly fitted onto and firmly attached to the ATP optical measuring instrument and may have, for example, a semi-elliptical cylindrical shape as illustrated in the drawings.

In addition, although not illustrated in the drawings, the mixing device may be more firmly fixed to the ATP optical measuring instrument by forming a screw hole passing through the coupling part 40 to the coupling groove 50, installing a tightening screw in the screw hole, fitting the coupling groove 50 onto the ATP optical measuring instrument, and tightening the tightening screw.

The battery 60 serves to supply power to the mixing module 30. Since the battery 60 is provided, portability of the mixing device can be secured. A general rechargeable lithium battery may be used as the battery 60. The battery 60 may be detachably attached to one side surface of the main body 10, for example, a side surface opposite to a side surface on which the coupling part 40 is disposed. The battery 60 may be connected to the mixing module 30 through an electric wire.

As described above, the mixing device according to the present invention may include the swab kit insertion part 20 into which the commercial ATP swab kit is inserted; the internal insertion type vibration (or sonicator or vortexing) module 30 which vibrates, sonicates, or vortexes the inserted swab kit to induce an effective occurrence of an ATP-luciferin reaction; and the coupling part 40 which may be detachably attached to the commercial ATP optical measuring instrument.

In addition, the present invention provides an ATP optical measuring system comprising an ATP optical measuring instrument; a swab; an ATP reaction swab kit; and the above-described mixing device.

The commercial ATP optical measuring instrument illustrated in FIG. 2 may be used as the ATP optical measuring instrument. The commercial ATP optical measuring instrument may include a swab kit insertion part into which a swab kit may be fitted, a detection part for measuring an intensity of light generated by an ATP-luciferin reaction, a display for displaying a measurement result, a case, a support, and the like and may further include a battery so that the commercial ATP optical measuring instrument can be used as a portable type.

The commercial swab kit illustrated in FIG. 2 may be used as the ATP reaction swab kit. The commercial swab kit may be formed as a test tube having a substantially cylindrical shape and may include an ATP extraction solution and a fluorescence reaction solution, and the like therein. The ATP extraction solution is for extracting ATP present in cells and may be, for example, a cell lysis buffer. The fluorescence reaction solution is for fluorescence reacting and may include, for example, luciferin, luciferase, and the like. In addition, the commercial swab kit may further include pure water (or alcohol or saline solution) for dilution, a buffer solution for pH neutralization, and the like.

A commercial swab may be used as the swab. The swab may include a support having a rod shape, a cotton ball wound around an end of the support, and the like and may be a sterilized product.

The mixing device is the above-described mixing device specifically manufactured according to the present invention.

In addition, the present invention provides an ATP optical measuring method using the ATP optical measuring system.

Specifically, first, the mixing device is attached to the ATP optical measuring instrument using the coupling part 40 of the mixing device.

Then, a sample is sampled using a commercial swab from a place where the presence or absence of bacteria is to be diagnosed.

Then, the swab with the sample thereon is put into the ATP reaction swab kit. Accordingly, an ATP-luciferin reaction starts in the swab kit.

Then, the swab kit into which the swab is put is inserted into the swab kit insertion part 20 of the mixing device, and the reactant in the swab kit is mixed using the mixing module 30. Accordingly, the ATP-luciferin reaction is activated to sufficiently occur in the swab kit.

Then, the swab kit in which the reaction has occurred by mixing is fitted into the swab kit insertion part of the ATP optical measuring instrument, after measuring an intensity of light generated by the ATP-luciferin reaction using the detection part, an RLU value (a unit that represents an intensity of light generated by the reaction), which is displayed on the display, is checked.

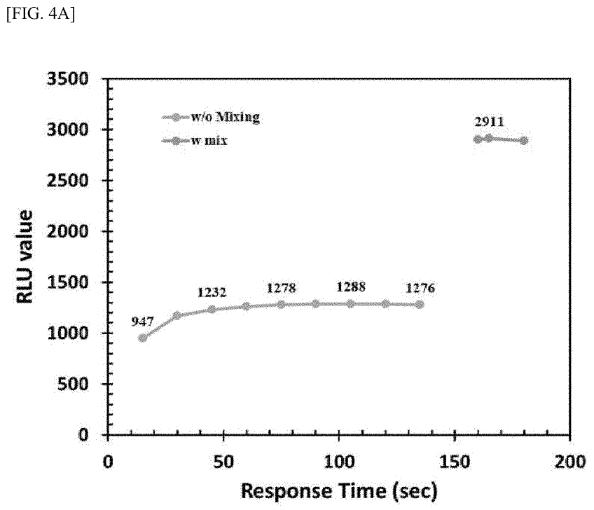

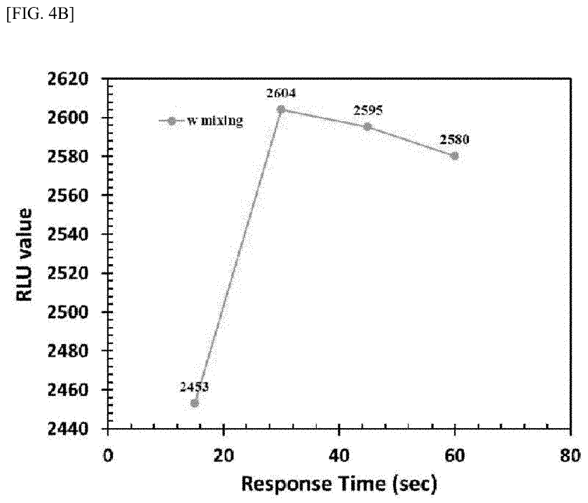

Referring to FIG. 4A, when the mixing is not performed, after the RLU value reaches the highest point, and if the mixing is sufficiently performed, then the highest point of the RLU value is greatly changed.

Referring to FIGS. 4A-B, in the same sample, when a case in which the mixing is not performed (see FIG. 4A) and a case in which the mixing is sufficiently performed (see FIG. 4B) are compared, in the case in which the mixing is performed, a time it takes for the RLU value to reach the highest point is 3 to 4 times faster than that in the case in which the mixing is not performed.

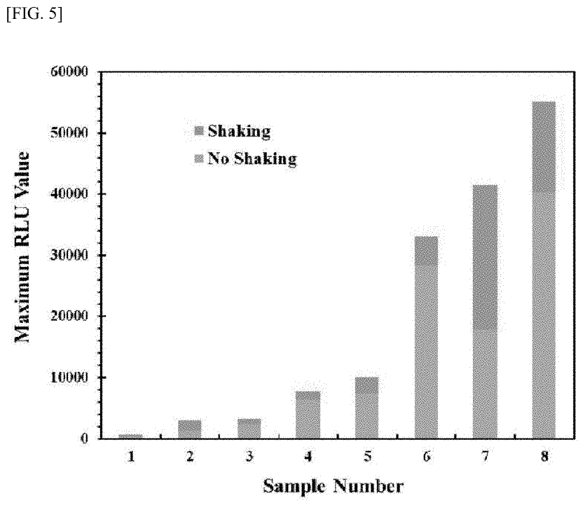

Referring to FIG. 5, in 8 samples having different concentrations, RLU values are checked using the commercial ATP optical measuring instrument without mixing, each of the RLU value reaches the highest point, and then, when the mixing is sufficiently performed, the highest RLU value is meaningfully changed.

When samples 6, 7, and 8 are compared, it may be seen that, when the mixing is not performed, RLU values having different levels from actual concentrations are measured.

Since the COVID-19 pandemic, interest in indoor/outdoor air quality and hygiene inspection is increasing worldwide, and needs for real-time quantitative monitoring technology for the indoor/outdoor air quality and hygiene inspection are increasing.

Among various technologies for monitoring air quality and hygiene inspection, an ATP optical measuring method using the ATP bioluminescence reaction can easily and rapidly detect a sample, and various commercial products have already been released.

Each of the products includes an ATP optical measuring instrument and a swab kit, and each method of using the products introduced by manufactures includes sampling a sample using a swab, manually shaking the swab kit for a reaction, and measuring an RLU value using the ATP optical measuring instrument. In this case, by using the vibration/sonicator/vortexing device detachable to the commercial ATP optical measuring instrument according the present invention instead of the method of manually shaking for a reaction, the consistent RLU value can be more rapidly obtained, and thus quantitative analysis is possible.

In addition, since the present invention can be also applied to an air quality monitoring method using an ATP optical measuring method, quantitative analysis for air quality can be rapidly and reproducibly performed.

As described above, with a trend in which the interest in indoor/outdoor air quality and hygiene inspection is increasing, it is expected that a demand for the commercial ATP optical measuring instrument will be increased, and marketability and expectation for the detachable vibration/sonicator/vortexing device according to the present invention which can be used together with the commercial ATP optical measuring instrument will be gradually increased.

What is claimed is:

1. A mixing device for an adenosine triphosphate (ATP) optical measuring instrument, comprising:
a main body;
a swab kit insertion part which is formed in the main body and provides an insertion space for an ATP reaction swab kit;
a mixing module which is disposed around a lower perimeter of the swab kit insertion part and mixes a reactant in the inserted swab kit; and
a coupling part disposed on one side surface of the main body and detachably attached to the ATP optical measuring instrument.

2. The mixing device of claim 1, wherein the mixing module is a vibration module.

3. The mixing device of claim 1, wherein the mixing module is a sonicator.

4. The mixing device of claim 1, wherein the mixing module is a vortexing module.

5. The mixing device of claim 1, wherein a speed of the mixing module is in the range of 3,000 to 5,000 rpm.

6. The mixing device of claim 1, wherein the coupling part includes a coupling groove to be fitted onto the ATP optical measuring instrument.

7. The mixing device of claim 1, further comprising a battery mounted on the main body.

8. An adenosine triphosphate (ATP) optical measuring system comprising:
an ATP optical measuring instrument;
a swab;
an ATP reaction swab kit; and
the mixing device of claim 1.

9. An adenosine triphosphate (ATP) optical measuring method comprising:
attaching the mixing device to the ATP optical measuring instrument of the ATP optical measuring system of claim 8;
sampling a sample using a swab from a place where the presence or absence of bacteria is to be diagnosed;
putting the swab with the sample thereon into the ATP reaction swab kit;
mixing after inserting the swab kit, into which the swab is put, into the mixing device;
fitting the mixed swab kit onto the ATP optical measuring instrument;
measuring an intensity of light generated by an ATP-luciferin reaction; and
checking a relative light unit (RLU) value which is a unit that represents an extent of the reaction.
